(19) United States
(12) Patent Application Publication
(10) Pub. No.: US 2022/0036608 A1
(43) Pub. Date: Feb. 3, 2022

(54) SYNTHETIC MAMMOGRAM WITH REDUCED OVERLAYING OF TISSUE CHANGES

(71) Applicant: Siemens Healthcare GmbH, Erlangen (DE)

(72) Inventors: Julia WICKLEIN, Erlangen (DE); Steffen KAPPLER, Effeltrich (DE)

(73) Assignee: Siemens Healthcare GmbH, Erlangen (DE)

(21) Appl. No.: 17/381,303

(22) Filed: Jul. 21, 2021

(30) Foreign Application Priority Data
Jul. 31, 2020 (DE) 10 2020 209 706.2

(51) Int. Cl. G06T 11/00; A61B 6/02; A61B 6/00

(52) U.S. Cl. CPC G06T 11/006; A61B 6/025; G06T 2210/41; A61B 6/5217; A61B 6/5223; A61B 6/502

(57) ABSTRACT

A method is for generating a first synthetic mammogram. In an embodiment, the method includes acquiring a tomosynthesis dataset including a plurality of projection images of a tissue region from different projection directions in a projection angle range; reconstructing a slice image dataset based on the tomosynthesis dataset; localizing tissue changes in the slice image dataset; determining a first projection direction for a first synthetic mammogram based on the spatial distribution of the tissue changes in the slice image dataset and generating the first synthetic mammogram in the first projection direction based on the tomosynthesis dataset.

### PRIORITY STATEMENT

The present application hereby claims priority under 35 U.S.C. § 119 to German patent application number DE 102020209706.2 filed Jul. 31, 2020, the entire contents of which are hereby incorporated herein by reference.

### FIELD

Example embodiments of the invention generally relate to a method for generating a first synthetic mammogram for improved detection of overlaying structures or lesions.

### BACKGROUND

Digital breast tomosynthesis (DBT) enables a three-dimensional imaging of the breast. A plurality of slices are reconstructed at different heights based on a plurality of acquired (x-ray) projections. Slice images of the breast are produced as a result. The projections are acquired at different angles within a limited angular range, for example in an angular range of substantially 50 degrees. In this case 25 projections may be acquired, for example.

An advantage of digital breast tomosynthesis compared to a full-field digital mammography (FFDM) scan is the possibility of resolving or separating overlapping tissue structures. Particularly advantageously, spiculated lesions can be detected in certain slices. In contrast thereto, in a full-field digital mammography scan, the lesion may be overlaid by other tissue structures or vessels from other slices, thus making a detection of lesions more difficult. Full-field digital mammography has in particular advantages in terms of speed of evaluation by the user and the visualization of microcalcification clusters. Accordingly, clinical protocols routinely comprise digital breast tomosynthesis as well as full-field digital mammography in order to combine the advantages of both imaging modalities. However, since roughly the same dose is applied both in digital breast tomosynthesis and in full-field digital mammography, combining both imaging modalities means that the patient dose is roughly doubled compared to a full-field digital mammography scan alone.

It is therefore desirable to calculate what is termed a synthetic mammogram from the acquired tomosynthesis dataset of the digital breast tomosynthesis scan. This enables an additional dose to be avoided or reduced while still retaining the advantages of two-dimensional full-field digital mammography.

The forward projection of a three-dimensional tomosynthesis volume onto a two-dimensional slice leads in turn to overlapping tissue and structures or lesions and as a result it is no longer possible to make the most of the advantages of digital breast tomosynthesis. This disadvantage can be prevented by applying what are known as computer-aided detection (CAD) methods. A CAD method is able to identify specific regions in the tomosynthesis volume that are of interest to the radiologist or user. The identified regions may be highlighted or marked in the synthetic mammogram in such a way that the identified regions are also visible in the two-dimensional view in the synthetic mammogram. At the same time the lesions may still overlap.

A further possibility is the use of a probability map based on a weighted subtraction of a high-energy and low-energy tomosynthesis dataset or a so-called dual-energy tomosynthesis dataset. This likewise enables regions of interest to be identified. The problem of two or more overlapping structures in the plane of the forward projection continues to exist, however.

A mammography method in which a simulated volume that represents a tissue region is rotated is known from the publication DE 10 2011 003 135 B4.

The indicated rotating synthetic mammogram corresponds to a plurality of two-dimensional synthetic mammograms that are calculated for different projection angles. By rotating or scrolling through the plurality of synthetic mammograms within the rotating synthetic mammogram, hidden or overlaying structures or lesions can be detected via the different viewing directions.

### SUMMARY

The inventors have discovered the problem that detecting overlaying structures or lesions in two-dimensional views is made more difficult, but at the same time that two-dimensional views are particularly advantageous for a rapid evaluation of the acquired images.

Embodiments of the invention disclose a method, a mammography system, a computer program product and a computer-readable data medium which enable an improved detection of overlaying structures or lesions in a two-dimensional synthetic mammogram.

Embodiments of the invention are directed to a method, a mammography system, a computer program product, and a computer-readable data medium.

An embodiment of the invention is directed to a method for generating a first synthetic mammogram, the method comprising acquisition, reconstruction, localization, determination, and generation. Mammography is one field of application of the method according to an embodiment of the invention.

An embodiment of the invention further relates to a mammography system for example, in an embodiment, performing a method according to an embodiment of the invention. The mammography system may comprise in particular an acquisition unit, a reconstruction unit, a localization unit, a determination unit and a generation unit. The mammography system is configured for generating a first synthetic mammogram. The mammography system may further comprise a display unit, for example a screen, and an input unit. The display unit may be embodied for example as a touch-sensitive screen which permits inputs by touching the screen.

An embodiment of the invention further relates to a computer program product comprising a computer program which can be loaded directly into a memory device of a control device of a mammography system, the computer program product having program sections for performing all steps of a method according to an embodiment of the invention when the computer program is executed in the control device of the mammography system.

An embodiment of the invention further relates to a computer-readable medium on which program sections are stored that can be read in and executed by a computer unit in order to perform all steps of a method according to an embodiment of the invention when the program sections are executed by the mammography system. Advantageously, the method according to an embodiment of the invention may be performed in particular automatically.

An embodiment of the invention further relates to a method for generating a first synthetic mammogram, comprising:

acquiring a tomosynthesis dataset including a plurality of projection images of a tissue region from different projection directions in a projection angle range;

reconstructing a slice image dataset based on the tomosynthesis dataset;

localizing tissue changes in the slice image dataset;

determining a first projection direction for a first synthetic mammogram based on a spatial distribution of the tissue changes in the slice image dataset; and

generating the first synthetic mammogram in the first projection direction based on the tomosynthesis dataset.

An embodiment of the invention further relates to a mammography system comprising:
a memory storing a computer program; and
at least one processor, upon executing the computer program, being configured to perform at least
   acquiring a tomosynthesis dataset including a plurality of projection images of a tissue region from different projection directions in a projection angle range;
   reconstructing a slice image dataset based on the tomosynthesis dataset;
   localizing tissue changes in the slice image dataset;
   determining a first projection direction for a first synthetic mammogram based on a spatial distribution of the tissue changes in the slice image dataset; and
   generating a first synthetic mammogram in the first projection direction based on the tomosynthesis dataset.

An embodiment of the invention further relates to a non-transitory computer program product storing a computer program, directly loadable into a memory device of a control device of a mammography system, the computer program including program sections for performing the method of an embodiment when the computer program is executed in the control device of the mammography system.

An embodiment of the invention further relates to a non-transitory computer-readable medium storing program sections, readable and executable by at least one processor to perform the method of an embodiment when the program sections are executed by the at least one processor.

### BRIEF DESCRIPTION OF THE DRAWINGS

Example embodiments of the invention are explained in more detail below with reference to drawings, in which:

FIG. 1 schematically shows a mammography system according to an embodiment of the invention;

FIG. 2 schematically shows a representation of a method according to an embodiment of the invention;

FIG. 3 schematically shows a view of a synthetic mammogram in a central projection direction;

FIG. 4 schematically shows a view of a synthetic mammogram in a projection direction PN;

FIG. 5 schematically shows a view of a first synthetic mammogram in a first projection direction; and

FIG. 6 schematically shows a view of a first synthetic mammogram subdivided into two slice images.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

An embodiment of the invention is directed to a method for generating a first synthetic mammogram, the method comprising acquisition, reconstruction, localization, determination, and generation. Mammography is one field of application of the method according to an embodiment of the invention.

In an embodiment, the acquisition includes a tomosynthesis dataset comprising a plurality of projection images of a tissue region is acquired from different projection directions in a projection angle range. The acquired projection images of the tissue region are generated by radiation emitted by an x-ray source, which radiation is detected by an x-ray detector after passing through the tissue region.

The tomosynthesis dataset comprises a plurality of projection datasets. A projection dataset is acquired for one projection direction. The x-ray source may for example be moved or pivoted along a circular segment. Alternatively, multiple x-ray emitters may be arranged for example along a circular arc or a straight line. The x-ray detector may preferably be arranged in a fixed or stationary manner. Alternatively, the x-ray detector may for example be moved or tilted counter to the movement of the x-ray source. The projection direction may be specified in particular by the direction of incidence of the central beam onto the x-ray detector or a spatial point in the examination subject, in this case preferably the breast. A central projection may be acquired for example at a projection angle of 0 degrees, in which case the projection direction may correspond to a surface normal of the detection surface.

The tissue to be examined, in particular the breast, may be positioned over the, in particular stationary, x-ray detector, the tissue to be examined preferably being compressed. The breast tissue may be compressed in a compression unit. The compression unit may for example comprise an upper compression paddle and a lower compression paddle. The lower compression paddle may be embodied for example by the top side of the x-ray detector or its housing.

The x-ray source may be pivoted in a number of increments or continuously, for example in a range of +/−25 degrees, and a plurality of two-dimensional x-ray images or projection datasets may be acquired from different pivot positions of the x-ray source or from different projection directions. In particular a stationary x-ray detector may be used in this case.

The x-ray source emits, in craniocaudal scans for example, x-ray beams from positions arranged along a line extending parallel to the shoulder-to-shoulder axis of a patient. By using a beam path parallel to the chest wall, it is possible to image the entire tissue of the breast and the thorax is not exposed to radiation. A three-dimensional image is then generated from the plurality of two-dimensional x-ray images via the reconstruction.

In the reconstruction step, a slice image dataset is reconstructed based on the tomosynthesis dataset or based on the plurality of acquired projection images. A slice image dataset is generated in the process.

The slice image dataset may be generated via a backprojection, in particular a filtered backprojection, iterative reconstruction or algebraic reconstruction based on the tomosynthesis dataset.

In the localization step, tissue changes are localized in the slice image dataset. A tissue change may be a change in tissue density, a calcified structure, a lesion, a so-called mass or a conspicuity, for example in the attenuation values. In particular, a three-dimensional probability map for tissue changes may be generated, in particular automatically. The tissue change may also be specified as a risk indicator for a malignancy. The tissue change may be determined for example via a machine-learning method, a neural network or/and a computer-aided detection (CAD) method. Identified tissue changes may be entered for example as a probability value in a three-dimensional probability map. The probability value may indicate a probability for the presence of malignant tissue. The probability map comprises for example the location, in particular via an x-y-z coordinate, and an extent or a spatial distribution of the tissue change.

In an embodiment, the determination includes determining a first projection direction for a first synthetic mammogram based on the spatial distribution of the tissue changes in the slice image dataset. The first projection direction may in particular be determined automatically. In particular, a first projection direction may be determined by which a particularly large number of tissue changes may be visualized separately from one another and not overlapping in a two-dimensional view. The first projection direction may also be referred to as the optimal projection direction. An improved overview of tissue changes may be visualized by way of the first projection direction. In particular, more tissue changes may be visualized in the first projection direction than in other projection directions. The first projection direction may be a preferred projection direction.

In an embodiment, the generation includes generating the first synthetic mammogram in the first projection direction based on the tomosynthesis dataset. The slice image dataset and/or the projection datasets of the tomosynthesis dataset may be used in this case for example. The synthetic mammogram is generated in the first projection direction. The first projection direction may be different from the central projection direction, for example 0 degrees.

In an embodiment, the calculation of the synthetic mammogram or of a synthetic projection may be performed in particular on the basis of the tomosynthesis dataset. Since the projection datasets are acquired with only a fraction of the dose for a full-field digital mammography scan, for example 1/25 of the dose in the case of 25 projections, the contrast-to-noise ratio suffers enormously for each individual projection. Furthermore, a smearing of the information may be expected due to the movement of the x-ray source.

Consequently, the use of a single projection image does not fulfill the requirements to be met by a scan in terms of satisfactory quality. A backprojection of the reconstructed volume data may be used in order to take the fullest possible account of the information from the tomosynthesis volume in the synthetic mammogram. For example, an average intensity projection (AIP), an average projection image, a maximum intensity projection (MIP) or, where appropriate, a high-interest projection (HIP) may be used as a basis. The projection dataset of the first projection direction may be used as a basis. Alternatively or in addition, further known methods for calculating a synthetic mammogram may be used.

The inventors have recognized that an ideal or optimal or optimized projection direction, in this case the first projection direction, may be used for the first two-dimensional synthetic mammogram in order to transfer the advantages of a rotating mammogram back into a two-dimensional visualization. This advantageously enables a reduced evaluation time, also called the “reading time”, to be achieved, in particular in screening applications or in comparisons with a previous acquisition of a previous examination. The previous acquisition may be for example a full-field digital mammography acquisition or a synthetic mammogram. The first synthetic mammogram may additionally comprise highlighted or marked structures transferred from a three-dimensional probability map for tissue changes.

The first projection direction may advantageously permit an improved resolution of tissue changes.

Advantageously, a first synthetic mammogram may be studied initially to obtain an overview of the examination, for example instead of a rotating mammogram or the view of many slices in succession. The evaluation time expended by the user may advantageously be shortened.

According to an embodiment of the invention, the first synthetic mammogram has a minimum overlap of tissue changes from different slices of the slice image dataset. The tissue changes may be distributed in particular in depth in the tissue under examination, i.e. distributed over multiple slices. The tissue changes may furthermore have different or similar spatial extents, both two-dimensionally within the slice plane and within one or more slice thicknesses. Overlaps of tissue changes may therefore be present in the projection along a projection direction. Due to the overlap, overlapping tissue changes cannot be separated. Due to the overlap, tissue changes may be covered or hidden by tissue changes in another slice. A maximization method may be applied for example in order to determine the first projection direction in which for example the most tissue changes are formed or the highest occupancy with tissue changes in terms of surface area is formed in the projection. A minimum overlap of tissue changes may be achieved. The diagnosis can advantageously be improved. Advantageously, the first synthetic mammogram may be used as an improved overview image, for example at the start of the evaluation.

According to an embodiment of the invention, a probability map for tissue changes is generated in the localization step. The probability map may also be referred to as a lesion probability map. Found or suspected tissue changes may be entered in an, in particular three-dimensional, probability map. The probability map may for example indicate a probability for a tissue change or for malignant tissue. The probability map may advantageously comprise the distribution of tissue changes in a depth-resolved manner in the tomosynthesis volume. The probability map may be understood as a volume for visualizing tissue changes.

The three dimensions of the probability map may extend for example in the slice plane and in the stacking direction of the slices. The probability values may be assigned to spatial points or voxels within the tomosynthesis volume. The coordinates of the spatial points or voxels may be specified in Cartesian coordinates.

According to an embodiment of the invention, multiple forward-projection datasets are generated in the determination step by way of forward projection of the probability map for a different projection direction in each case. A minimum or reduced overlap of tissue changes in the two-dimensional view may be achieved for example by way of a forward projection of a probability map for tissue changes or for malignant regions. The overlap may also be referred to as an overlapping, masking or overlay. The overlap may relate in particular to a two-dimensional view. The, in particular three-dimensional, probability map may be mapped by way of forward projection in a projection direction into a forward-projection dataset. Correspondingly different forward-projection datasets may be generated for different projection directions. Advantageously, different forward-projection datasets or different projection directions may be compared in terms of the distribution of tissue changes in the projection.

For example, a forward-projection dataset may be determined for each projection direction of the tomosynthesis dataset acquisition. For example, a forward-projection dataset may be determined for a plurality of projection directions of the tomosynthesis dataset acquisition. For example, a forward-projection dataset may be determined for each second projection direction. The number of forward-projection datasets to be determined may for example depend on the total number of tissue changes in the probability map. The more tissue changes are recorded in the probability map, the more projection directions may be referred to.

According to an embodiment of the invention, a parameter value based on the planar distribution of the probability values for tissue changes in the forward-projection dataset is determined for a plurality of projection directions.

A parameter or a parameter value may be determined for the purpose of a comparison of the projection directions in terms of the distribution of the tissue changes. The parameter may for example be chosen as the same for all examinations. Alternatively, the parameter may for example be chosen as a function of the number of tissue changes in the probability map.

A parameter for determining an optimal or first projection direction may for example be the number of connected components or elements within the two-dimensional view or the forward-projection dataset. The number of connected elements in the forward-projected image and consequently the projection direction may be referred to as optimal when the number of connected elements is close to the number of tissue changes in the three-dimensional probability map.

A parameter for determining an optimal or first projection direction may for example be the number of highlighted or marked pixels within the two-dimensional view or the forward-projection dataset. The optimal or first projection direction may be determined by the maximization of the number of highlighted or marked pixels in the forward-projection dataset or in the synthetic mammogram. The planar distribution may be specified in an occupancy of image elements with the information relating to a tissue change. The planar distribution may specify a surface area metric for tissue changes in the projection.

The parameter value of the parameter may be in particular a natural or real positive number. A number of parameters may be combined, for example into one parameter and correspondingly into a common parameter value.

The parameter and its parameter value may be in particular a measure for tissue changes, in particular the number of regions having at least one tissue change, or the number of tissue changes, the number of lesions, the number of connected elements or tissue changes, the surface area of the tissue changes in the projection or the number of highlighted image elements or pixels in the projection. Advantageously, an optimal projection direction may be determined automatically by way of a numerical value.

According to an embodiment of the invention, the projection direction having the maximum parameter value is determined as the first projection direction in the determination step. The projection direction to which the maximum parameter value is assigned may be determined as the first projection direction.

The parameter values for the different projection directions may be compared. Ideally, a maximum parameter value may be determined. The projection direction having the maximum parameter value may be embodied in particular as the optimal projection direction in order to provide an enhanced visualization of the tissue changes. The projection direction to which the maximum parameter value is assigned may be chosen as the first projection direction. Advantageously, an optimal projection direction may be determined in a simplified manner. The optimal projection direction or first projection direction may in particular be determined automatically.

According to an embodiment of the invention, the determined parameter values are compared with one another, and in the case of at least two parameter values in the range between the maximum determined parameter value and 90 percent of the maximum determined parameter value, that projection direction which is disposed closest to a central projection direction is chosen as the first projection direction. In the case of a further parameter value in relation to a further projection direction different from the first projection direction having a deviation of up to 10 percent from a maximum parameter value, that projection direction closest to the central projection direction may for example be chosen as the first projection direction.

If two or more parameter values including the maximum parameter value lie in a narrow value range, then in principle the assigned projection directions may be suitable or optimal. If two or more parameter values including the maximum parameter value lie in a narrow value range, then a projection direction may be selected, in particular automatically, as the first projection direction from the projection directions that are assigned to the parameter values.

In the case of a number of optimal or suitable projection angles, the projection direction lying closest to the central projection direction or to the projection direction at an x-ray source setting of 0 degrees may be chosen. Advantageously, artifacts, for example smearing artifacts, may be reduced at a projection direction around approx. 0 degrees or in the central projection direction.

According to an embodiment of the invention, an overlap parameter for the overlapping of tissue changes is determined from different slices of the slice image dataset.

The overlap parameter may for example comprise a surface area or a number of contiguous image elements containing information relating to at least one tissue change. The information concerning the at least one tissue change may have its origin in different slices of the slice image dataset. A larger surface area may be a pointer to a number of overlapping, in particular partially overlapping, tissue changes in the projection. For example, an average-sized surface area of the tissue changes for one forward-projection dataset or for a plurality of projection datasets may be determined, in particular individually, preferably collectively. In the case of a deviation of, for example, at least a factor 2 from the average value, this may relate either to a large tissue change or to an overlapping of several tissue changes. If a deviation from the average value, for example a factor 2, is to be observed in one projection direction only, this may be indicative of an overlap. The threshold value may relate for example to a deviation from the average value. In the above example, the threshold value would be factor 2 of the average value. The threshold value may relate for example to a size of the surface area.

The overlap parameter may comprise a number of tissue changes in the forward-projection datasets. A number of tissue changes or contiguous elements may be determined for a number of projection directions in the forward-projection dataset in each case. The number of tissue changes to be expected may correspond to a predetermined value according to the probability map.

The number to be expected may correspond to the number of tissue changes in the probability map. A fluctuation in the number of tissue changes in the forward-projection datasets as a function of the projection direction may be a pointer to an overlapping of tissue changes. A lower number may be indicative of an overlap. A higher number may be indicative of a minor overlap. A threshold value may be specified based on the expected number, for example via a percentage deviation. A threshold value may be predetermined. The threshold value may be based for example on statistical values. The threshold value may be based for example on the compressed breast thickness.

A projection direction having a number close to the expected number may in particular correspond to the first projection. The threshold value may be specified based on a deviation or difference based on the expected number and the number in the forward-projection dataset. A difference of 2 may be specified as the threshold value, for example.

Within the scope of an embodiment of the forward projection, image elements having an entry of more than one tissue change, in particular from different slices, from the probability map may be marked with an overlap flag. The threshold value may in this case be specified by way of the number or the spacing of the contributing slices. The threshold value may thus be a spacing of a slice, for example. A tissue change may extend over multiple slices. For the threshold value in terms of a number of contributing slices, reference may be made to a statistically or empirically known value in respect of the extent of tissue changes.

A forward projection of a binary probability map may be generated in which each tissue change is reduced to a slice having maximum extent. This enables a two-dimensional map to be generated in which all entries greater than 1 point to overlapping of multiple tissue changes. For the central projections, this can be made possible with reduction to one slice in the z-direction. For the outer projections, this slice can be placed parallel to the corresponding projection direction in order to avoid errors at the boundary regions of the tissue changes.

Advantageously, an overlap or an overlaying of tissue changes may be quantified. Advantageously, the user may be alerted to an overlap, for example.

According to an embodiment of the invention, if an overlap parameter exceeds a threshold value, the first synthetic mammogram is subdivided into two slice images. The threshold value may in particular be defined in such a way that a pointer to an overlap may be present if a threshold value is exceeded. If the threshold value is exceeded by the overlap parameter, two so-called thick slices can be generated for the first synthetic mammogram. The first synthetic mammogram may therefore be subdivided into two slices. The two slices may in particular represent the tomosynthesis volume divided in half. Alternatively, the slice thickness may be different for the two slices, for example as a function of the density or distribution of tissue changes in the probability map in relation to the depth in the examination subject.

More than one synthetic two-dimensional image may therefore be generated, in particular in the central projection direction. If there is a pointer to a particularly high number of tissue changes, the central projection direction may preferably be chosen or determined as the first projection direction.

The subdivision into at least two slice images may therefore correspond to a reconstruction of a tomosynthesis volume having very thick and at the same time few slices. The number of slices may in this case be chosen in particular as minimal so that overlapping structures or tissue changes are reduced or preferably avoided. Advantageously, the evaluation of images having in particular a large number of tissue changes can be simplified.

According to an embodiment of the invention, if an overlap parameter exceeds a threshold value, a first synthetic mammogram is generated in a first projection direction and a second synthetic mammogram in a second projection direction that is different from the first projection direction. The first and second projection directions may preferably be as far apart as possible from one another. For example, the first and the second synthetic mammogram may be generated for the projection directions spaced at a maximum distance apart, for example −25 degrees and +25 degrees. The first and the second synthetic mammogram may be generated for example for suitable projection directions at a minimum spacing of 5 or 10 degrees. The first and the second synthetic mammogram may be generated for example for the central projection direction and a suitable projection direction. The first and the second synthetic mammogram may be generated for example for optimal projection directions spaced at a minimum distance apart.

In the event that no clear optimal projection direction for resolving all overlapping tissue changes can be found, two synthetic two-dimensional images may be generated. Advantageously, the evaluation of images having in particular a large number of tissue changes can be simplified.

According to an embodiment of the invention, the tissue change in the first synthetic mammogram and/or in the second synthetic mammogram is highlighted or marked. The tissue changes may be highlighted or marked, in particular in color, in the first and/or second synthetic mammogram according to the probability map. The marking may be realized for example via a symbol or a border around the tissue change. From a tissue change in the first and/or second synthetic mammogram, a navigation into the slice image or the slice of the tomosynthesis volume containing the tissue change may be effected for example by way of selection or clicking on a display unit or alternatively in an automatic workflow. Advantageously, the evaluation of the tomosynthesis scan can be simplified.

According to an embodiment of the invention, the method further comprises the step of displaying at least one of the following:

a first synthetic mammogram,

a first synthetic mammogram with highlighting or marking of tissue changes,

in conjunction with at least one of the following:
a synthetic mammogram in a central projection direction,
a synthetic mammogram in a central projection direction with highlighting or marking of tissue changes, and
a slice image dataset.

The central projection direction may also be referred to as the principal projection direction. The following scenarios may be provided as a display sequence, for example:

first synthetic mammogram, tomosynthesis volume as stack of slice images;

synthetic mammogram in the central projection direction (in particular 0 degrees), first synthetic mammogram, tomosynthesis volume as stack of slice images;

first synthetic mammogram with highlighting or marking of tissue changes, tomosynthesis volume as stack of slice images;

synthetic mammogram in the central projection direction (in particular 0 degrees) with highlighting or marking of tissue changes, first synthetic mammogram with highlighting or marking of tissue changes, tomosynthesis volume as stack of slice images;

first synthetic mammogram, first synthetic mammogram with highlighting or marking of tissue changes, tomosynthesis volume as stack of slice images;

synthetic mammogram in the central projection direction (in particular 0 degrees), synthetic mammogram in the central projection direction (in particular 0 degrees) with highlighting or marking of tissue changes, first synthetic mammogram, synthetic mammogram in the central projection direction (in particular 0 degrees) with highlighting or marking of tissue changes, first synthetic mammogram with highlighting or marking of tissue changes, tomosynthesis volume as stack of slice images.

The findings or tissue changes in the images may be visualized in the scenarios in each case. A navigation into the tomosynthesis volume or slice image having the tissue change may be accomplished by selection of the tissue change or by clicking on the tissue change. In the event of an, in particular first, synthetic mammogram being generated with and without highlighting or marking of the tissue changes, it is possible to toggle smoothly between the two versions. The detection of tissue changes can advantageously be improved.

An embodiment of the invention further relates to a mammography system for example, in an embodiment, performing a method according to an embodiment of the invention. The mammography system may comprise in particular an acquisition unit, a reconstruction unit, a localization unit, a determination unit and a generation unit. The mammography system is configured for generating a first synthetic mammogram. The mammography system may further comprise a display unit, for example a screen, and an input unit. The display unit may be embodied for example as a touch-sensitive screen which permits inputs by touching the screen.

The acquisition unit may be configured for acquiring a tomosynthesis dataset. The acquisition unit is configured in particular for acquiring a plurality of projection images of a tissue region from different projection directions in a projection angle range. The acquisition unit may in particular comprise an x-ray source that can be pivoted in the projection angle range and an associated x-ray detector.

The reconstruction unit may be configured for reconstructing a slice image dataset based on the tomosynthesis dataset. The localization unit may be configured for localizing tissue changes in the slice image dataset. The determination unit may be configured for determining a first projection direction for a first synthetic mammogram based on the spatial distribution of the tissue changes in the slice image dataset. And the generation unit may be configured for generating the first synthetic mammogram in the first projection direction based on the tomosynthesis dataset.

Advantageously, the method according to an embodiment of the invention may be performed by the mammography system. The units of the mammography system may in particular be connected to one another, in particular by way of a direct, physical connection in the form of a cable connection or via a possibly wireless network connection.

An embodiment of the invention further relates to a computer program product comprising a computer program which can be loaded directly into a memory device of a control device of a mammography system, the computer program product having program sections for performing all steps of a method according to an embodiment of the invention when the computer program is executed in the control device of the mammography system.

An embodiment of the invention further relates to a computer-readable medium on which program sections are stored that can be read in and executed by a computer unit in order to perform all steps of a method according to an embodiment of the invention when the program sections are executed by the mammography system. Advantageously, the method according to an embodiment of the invention may be performed in particular automatically.

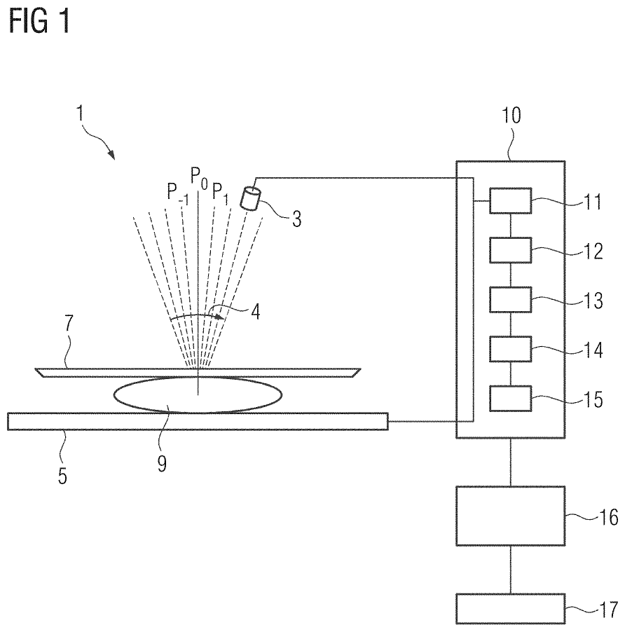

FIG. 1 shows an example embodiment of the mammography system 1 according to the invention. The mammography system 1 comprises a pivotable x-ray source 3 that is associated with an x-ray detector 5. The examination subject 9 or the breast is arranged on a surface of the x-ray detector 5 such that the surface of the x-ray detector 5 serves as a lower compression paddle. The examination subject is compressed between an upper compression paddle 7 and the x-ray detector 5. The x-ray source 3, the x-ray detector 5 and the upper compression paddle 7 are connected to the acquisition unit 11.

The projection directions . . . P₋₁, P₀, P₁ . . . , which can lie in a projection angle range 4 of −25 degrees to 25 degrees, for example, can be set by pivoting the x-ray source 3 relative to the examination subject 9 or the x-ray detector 5.

The control device or computer unit 10 comprises the acquisition unit 11, the reconstruction unit 12, the localization unit 13, the determination unit 14 and the generation unit 15. A display unit 16 and an input unit 17 are connected to the computer unit 10.

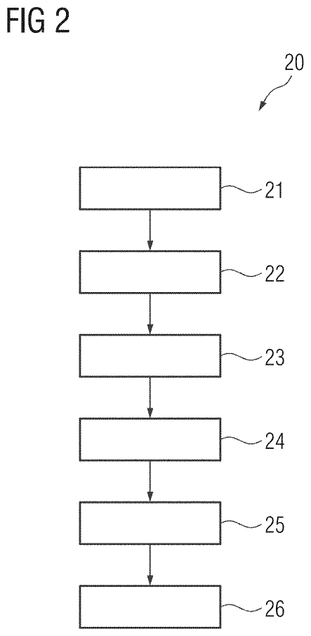

FIG. 2 shows by way of example a schematic representation of the inventive method 20 for generating a first synthetic mammogram. The method 20 comprises the steps of acquisition 21, reconstruction 22, localization 23, determination 24 and generation 25. The method 20 may further comprise the step of displaying 26.

In the acquisition step 21, a tomosynthesis dataset having a plurality of projection images of a tissue region is acquired from different projection directions in a projection angle range. In the reconstruction step 22, a slice image dataset is reconstructed based on the tomosynthesis dataset. In the localization step 23, tissue changes are localized in the slice image dataset. In the determination step 24, a first projection direction for a first synthetic mammogram is determined based on the spatial distribution of the tissue changes in the slice image dataset. In the generation step 25, the first synthetic mammogram is generated in the first projection direction based on the tomosynthesis dataset. The first synthetic mammogram may be displayed in the display step 26.

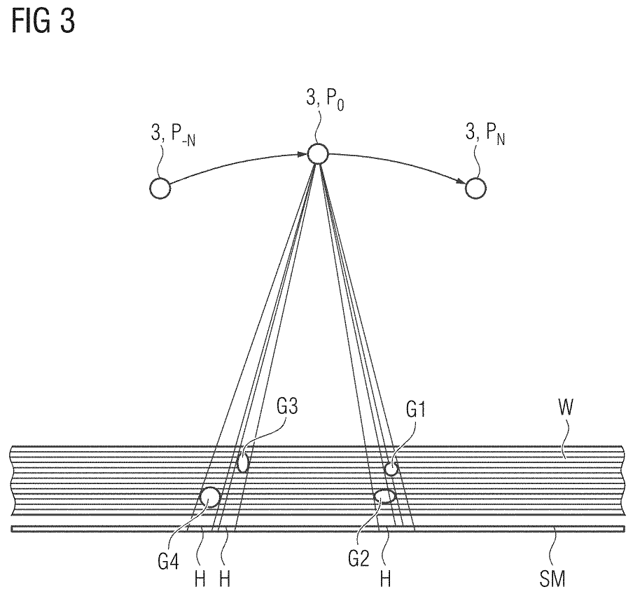

FIG. 3 shows by way of example a view of a synthetic mammogram SM in a central projection direction P₀. The probability map W containing the plurality of slices includes the tissue changes G1,G2,G3,G4. The probability map W can be represented in the slices of a tomosynthesis volume. The tissue changes may also be referred to as “regions of interest”. The probability map is forward-projected in the central projection direction P₀. Three connected elements are shown in the projection onto the detector plane. In the central projection direction P₀, the tissue changes G1,G2,G3,G4 are thus imaged accordingly onto the highlighted areas H in the synthetic mammogram SM. The tissue changes G3,G4 are imaged in separate highlighted areas H. The tissue changes G1,G2 are imaged as connected elements in a common highlighted area H. The tissue changes G1,G2 are therefore imaged inseparably in the synthetic mammogram SM although they are formed at different depths of the probability map. There is therefore an overlap of the tissue projections G1,G2 present in the synthetic mammogram SM, i.e. two tissue changes G1,G2 overlap in the central projection direction P₀.

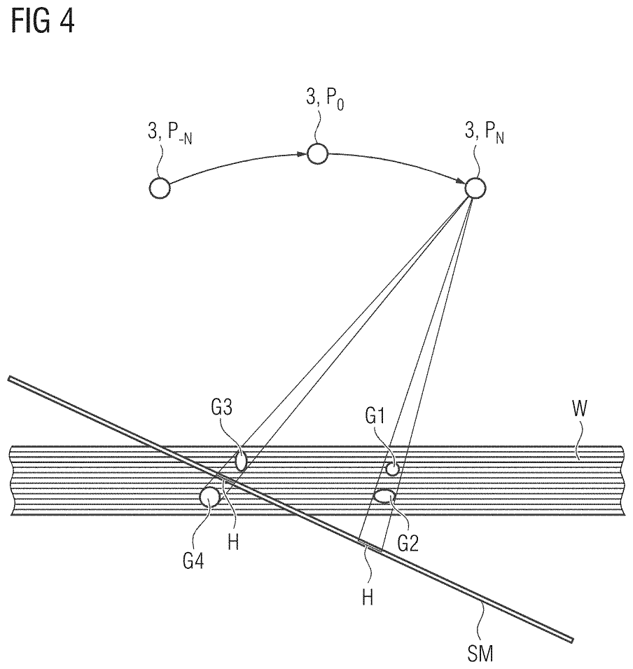

FIG. 4 shows by way of example a view of a synthetic mammogram in a projection direction PN. The probability map W is identical to the example in FIG. 3. The probability map is forward-projected in the projection direction PN. The projection direction PN is 25 degrees, for example. For the projection direction PN, in the forward projection only two connected elements can still be recognized or differentiated as highlighted areas H. The tissue changes G1,G2 and the tissue changes G3,G4 overlap in each case, with the result that these are represented as not separable in the synthetic mammogram SM.

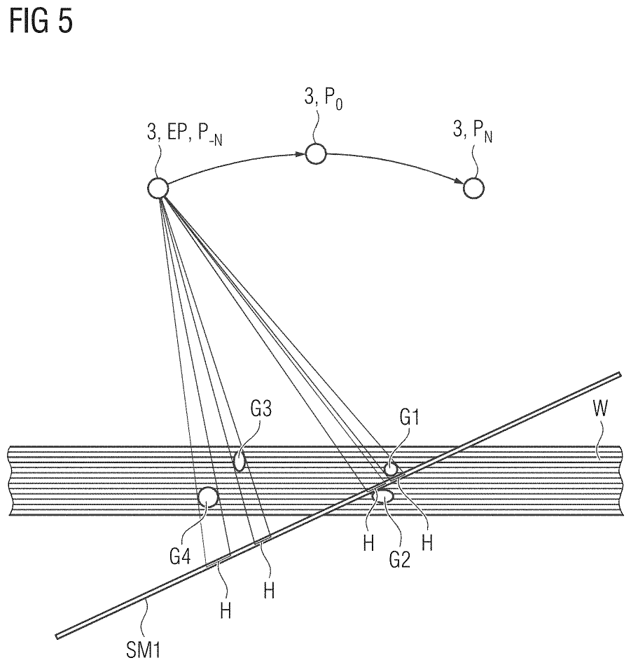

FIG. 5 shows by way of example a view of a first synthetic mammogram in a first projection direction EP. The probability map W is identical to the example as shown in FIGS. 3 and 4. The first projection direction EP is by way of example −25 degrees and therefore corresponds in this example to the projection direction P-N. The first synthetic mammogram SM1 shows four connected elements in highlighted areas H. The tissue changes G1,G2,G3,G4 can therefore be resolved individually. There is no overlap present.

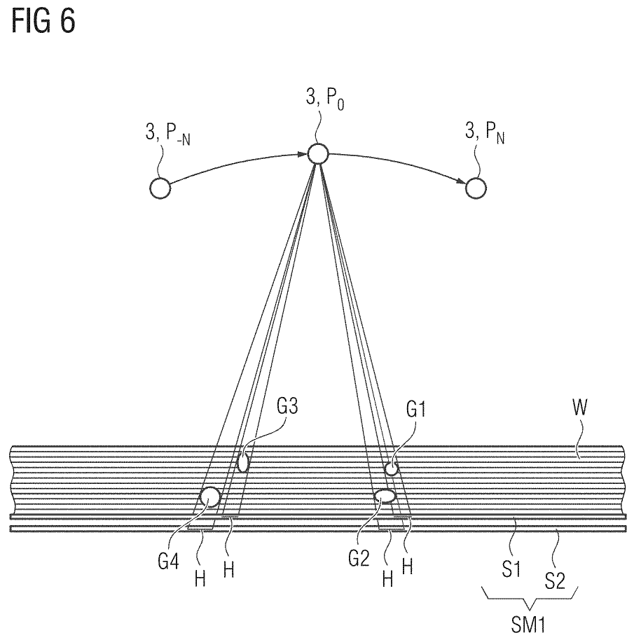

FIG. 6 shows by way of example a view of a first synthetic mammogram SM1 subdivided into two slice images S1,S2. The tissue changes G1,G2 would overlap as shown in FIG. 3. In order to avoid this, two slice images S1,S2 are generated for the first synthetic mammogram SM1. A top half of the probability map W or of the tomosynthesis volume is imaged in the slice image S1. A bottom half of the probability map W or of the tomosynthesis volume is imaged in the slice image S2. Thus, the tissue change G1 is imaged in the slice image S1, and the tissue change G2 in the slice image S2. The tissue changes G1,G2 may therefore be visualized as separated.

Although the invention has been illustrated in greater detail on the basis of the preferred example embodiment, the invention is not limited by the disclosed examples and other variations may be derived herefrom by the person skilled in the art without leaving the scope of protection of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for generating a first synthetic mammogram, comprising:
acquiring a tomosynthesis dataset including a plurality of projection images of a tissue region from different projection directions in a projection angle range;
reconstructing a slice image dataset based on the tomosynthesis dataset;
localizing tissue changes in the slice image dataset;
determining a first projection direction for a first synthetic mammogram based on a spatial distribution of the tissue changes in the slice image dataset; and
generating the first synthetic mammogram in the first projection direction based on the tomosynthesis dataset.

2. The method of claim 1, wherein the first synthetic mammogram has a minimum overlap of tissue changes from different slices of the slice image dataset.

3. The method of claim 1, wherein a probability map for tissue changes is generated during the localizing.

4. The method of claim 3, wherein a plurality of forward-projection datasets are generated during the determining by way of a respective forward projection of the probability map for each respective different projection direction.

5. The method of claim 4, wherein a parameter value is determined for a plurality of projection directions based on the planar distribution of the probability values for tissue changes in the forward-projection dataset.

6. The method of claim 5, wherein the projection direction including a maximum parameter value is determined as the first projection direction during the determining.

7. The method of claim 5, wherein the parameter values determined are compared with one another, and upon at least two parameter values in a range between the maximum determined parameter value and 90 percent of the maximum determined parameter value, the projection direction disposed relatively closest to a central projection direction is chosen as the first projection direction.

8. The method of claim 1, wherein an overlap parameter for the overlapping of tissue changes is determined, during the determining, from different slices of the slice image dataset.

9. The method of claim 8, wherein upon the overlap parameter exceeding a threshold value, the first synthetic mammogram is subdivided into two slice images.

10. The method of claim 8, wherein upon the overlap parameter exceeding a threshold value, a first synthetic mammogram is generated in a first projection direction during the generating and a second synthetic mammogram is generated in a second projection direction during the generating, the second projection direction being different from the first projection direction.

11. The method of claim 8, wherein the tissue change is highlighted or marked in at least one of the first synthetic mammogram and the second synthetic mammogram.

12. The method of claim 1, further comprising displaying at least one of:
a first synthetic mammogram, and
a first synthetic mammogram with highlighting or marking of tissue changes;
in conjunction with at least one:
a synthetic mammogram in a central projection direction,
a synthetic mammogram in a central projection direction with highlighting or marking of tissue changes, and
a slice image dataset.

13. A mammography system comprising:
a memory storing a computer program; and
at least one processor, upon executing the computer program, being configured to perform at least
acquiring a tomosynthesis dataset including a plurality of projection images of a tissue region from different projection directions in a projection angle range;
reconstructing a slice image dataset based on the tomosynthesis dataset;
localizing tissue changes in the slice image dataset;
determining a first projection direction for a first synthetic mammogram based on a spatial distribution of the tissue changes in the slice image dataset; and
generating a first synthetic mammogram in the first projection direction based on the tomosynthesis dataset.

14. A non-transitory computer program product storing a computer program, directly loadable into a memory device of a control device of a mammography system, the computer program including program sections for performing the method of claim 1 when the computer program is executed in the control device of the mammography system.

15. A non-transitory computer-readable medium storing program sections, readable and executable by at least one processor to perform the method of claim 1 when the program sections are executed by the at least one processor.

16. The method of claim 2, wherein a probability map for tissue changes is generated during the localizing.

17. The method of claim 16, wherein a plurality of forward-projection datasets are generated during the determining by way of a respective forward projection of the probability map for each respective different projection direction.

18. The method of claim 2, wherein an overlap parameter for the overlapping of tissue changes is determined, during the determining, from different slices of the slice image dataset.

19. The method of claim 18, wherein upon the overlap parameter exceeding a threshold value, the first synthetic mammogram is subdivided into two slice images.

20. The method of claim 18, wherein upon the overlap parameter exceeding a threshold value, a first synthetic mammogram is generated in a first projection direction during the generating and a second synthetic mammogram is generated in a second projection direction during the generating, the second projection direction being different from the first projection direction.
